(19) United States
(12) Patent Application Publication
(10) Pub. No.: US 2009/0163366 A1
(43) Pub. Date: Jun. 25, 2009

(54) TWO-PRIMER SEQUENCING FOR HIGH-THROUGHPUT EXPRESSION ANALYSIS

(75) Inventors: Elizabeth Nickerson, Reading, MA (US); Marie Sutherlin Causey, Belmont, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, Cambridge, MA (US)

(21) Appl. No.: 11/964,002

(22) Filed: Dec. 24, 2007

(51) Int. Cl. C40B 20/04; C07H 21/04

(52) U.S. Cl. 506/4; 536/24.33

(57) ABSTRACT

The disclosure provides a method of sequencing a nucleic acid molecule that contains two or more target regions to be sequenced (such as, for example, barcodes). The invention is advantageous for sequencing by synthesis two or more target regions whose combined lengths plus the length of any intermediate sequence exceeds the available read length on a given sequencing platform. The methods of the invention utilize nucleic acid constructs containing at least the following elements: a complement of a first universal primer, a first target sequence, an optional polynucleotide spacer, a complement of a second universal primer, and a second target sequence. A first round of sequencing by synthesis is performed to sequence the first target sequence by elongating the first universal primer. Once the sequence of the first target region is obtained, and before the complement of the second primer is reached, the first round of sequencing is terminated. Thereafter, a second round of sequencing by synthesis is initiated—this time, by elongating the second universal primer, thereby sequencing the second target region.

### TECHNICAL FIELD

The invention is in the field of molecular biology and relates to methods for nucleic acid analysis. In some aspects, the invention relates to methods of high-throughput gene expression analysis, particularly, in the context of sequencing by synthesis.

### BACKGROUND

Gene expression signatures comprised of tens of genes have been found to be predictive of disease type and patient response to therapy, and have been informative in countless experiments exploring biological mechanisms. High-density DNA microarrays are currently the method of choice for transcriptome analysis and represent a semi-quantitative route to signature discovery. However, gene expression signatures with diagnostic potential must be validated in large cohorts of patients, in whom measuring the entire transcriptome is neither necessary nor desirable. Perhaps more important is that the ability to describe cellular states in terms of a gene expression signature raises the possibility of performing high-throughput, small-molecule screens using a signature of interest as a read-out. For this to be practical, one would need to be able to screen thousands of compounds per day at a cost dramatically below that of conventional microarrays.

High-throughput genomic signature screening has been hampered by the lack of ability to quantitatively measure cellular changes in a reproducible, high-throughput manner. Since the sequencing of the human genome, new sequencing technologies have emerged that are capable of directly reading the individual sequences of single molecules of DNA or RNA, thus allowing the researchers to directly quantify the copy number for any individual gene or RNA of interest. With the advent of these high-throughput sequencing technologies, the researchers may now use quantitative RNA measurements from cell-based assays, across very large numbers of compounds, while monitoring changes in tens of thousands of genes.

Nevertheless, multiplexed high-throughput sequencing still remains constrained in complexity (number of samples sequenced in parallel) and in capacity (number of sequences obtained per sample). Physical space segregation of the sequencing platform into a fixed number of channels allows only limited multiplexing. Furthermore, all currently available high-throughput sequencing platforms show a trade-off between the average sequence read length and the number of nucleic acid molecules being sequenced.

One approach that overcomes the above limitations, is a high-information-content ‘barcoding’ in which each sample is associated with two or more uniquely designed nucleotide barcodes (unique sequence identifiers). The barcodes allow for independent samples to be pooled together for sequencing, with subsequent bioinformatic segregation of the sequencer output. ‘Barcodes’ have been used in several experimental contexts, for example, in sequence-tagged mutagenesis (STM) screens, where a sequence barcode acts as an identifier or type specifier in a heterogeneous cell-pool or organism-pool. STM barcodes are usually 20-60 nucleotides long, are selected or follow ambiguity codes, and are present as one unit or split into groups. Long barcodes, however, are not ideally suitable for use with available sequencing platforms with short read lengths (<30-50 bases). Although several groups have reported the use of very short (2- or 4-nt) barcodes, such short barcodes do not provide sufficient range of sample assignment and/or multiplexing that is required when tens to hundreds of thousands of samples need to be analyzed per run.

In the sequence-by-sequencing platforms with true single molecule sequencing (tSMS™; Helicos BioSciences, Cambridge, Mass.), the nucleic acids to be sequenced are hybridized to primers that are covalently attached to a derivatized glass surface so that the resulting primer/target duplexes are individually optically resolvable (i.e., they can detected as individual molecules). After a wash step, one or more optically labeled nucleotides is/are added along with a polymerase in order to allow template-dependent sequencing-by-synthesis to occur. The process is repeated until a sufficient number of target nucleotides is determined. Sequencing may be conducted such that a single labeled species of nucleotides is added sequentially, or multiple species with different labels, are added at the same time. tSMS™ systems currently provide read lengths on the order of 25 bases, which should be enough to sequence at least two barcodes of optimal length (10-15 nt). However, properly pasting two barcodes together (e.g., a well barcode and a gene barcode) requires an intervening hybridization site, which further adds 15-25 nucleotides between the barcodes, readily exceeding the available read length. An alternative approach that eliminates the intervening hybridization site requires a dramatically larger number of unique primers (e.g., 384 vs. 384,000), and therefore, is not practical. The current solution for reading two or more barcodes on tSMS™ systems, is to use a “melt-and-resequence” procedure (e.g., as described in U.S. Pat. No. 7,283,337). Melt-and-resequence requires template copying, strand melting and re-hybridization with a second primer, and the efficiencies of each step may be lower than desirable while variability, higher.

Accordingly, a need exists for new methods of rapid and cost-effective high-throughput gene expression analysis, including methods that utilize nucleic acid barcoding.

### SUMMARY OF THE INVENTION

The present invention provides a method of sequencing a nucleic acid molecule that contains two or more target regions to be sequenced (such as, for example, barcodes). The invention is advantageous for sequencing by synthesis two or more target regions whose combined lengths plus the length of any intermediate sequence exceeds the available read length on a given sequencing platform. This approach is suitable, for example, for reading nucleic acid barcodes. However, it may also be used for any other sequencing-by-synthesis application that requires sequencing any two or more non-contiguous regions (referred to herein as “target regions” or “target sequences”) within the same nucleic acid template. By designing nucleic acid constructs in such a way as to have a different universal primer site for each target region, the need for the “melt-and-resequence” procedure is obviated, resulting in increased efficiency, accuracy, and/or speed of nucleic acid identification. One of the applications for which the present invention is suitable is a genomic signature sequencing (GSS™) assay.

The invention utilizes nucleic acid constructs containing at least the following elements i) through v), arranged in the recited order in the 3′-to-5′ direction:

i) a complement of a first universal primer,

ii) a first target sequence,

iii) a polynucleotide spacer (optional),

iv) a complement of a second universal primer, and

v) a second target sequence.

In some embodiments, the first target sequence includes a sample-specific barcode sequence which identifies the source of the sample (e.g., position of sample on the plate, plate number, different treatment conditions, disease, tissue, etc.); and the second target sequence includes a gene-specific barcode identifying the gene of interest.

In general, the methods of the invention include at least the following steps. First, a plurality (e.g., 96, 384, 1536 or more) of biological samples is obtained, for example, for high throughput screening gene expression (GE-HTS) analysis. Each of the samples contains a plurality (e.g., 10, 100, 1000 or more) of nucleic acid constructs (“templates” or “template nucleic acids”) as described above. The samples are prepared for nucleic acid sequencing by synthesis. Then, a first round of sequencing by synthesis is performed to obtain the first target sequence by extending the complementary chain starting from the first universal primer. Once the sequence of the first target region is obtained, and before the complement of the second primer is reached, the first round of sequencing is terminated. The termination may be accomplished by an addition of a chain-terminating nucleotide to the reaction. Thereafter, a second round of sequencing by synthesis is initiated—this time, by elongating the second universal primer, thereby sequencing the second target region. To perform the above-recited steps, the following order of primer addition may be used, for example. Initially, the first universal primer is hybridized to a plurality of template nucleic acid molecules. For example, the first universal primer may be attached to the surface via the 5′-end, and 3′-OH being free, and the template nucleic immobilized onto the solid support via hybridization to the surface attached primer. After performing sequencing by synthesis from the first primer and incorporating a chain-terminating nucleotide, the second universal primer is hybridized to some of the plurality of templates. Subsequently, sequencing by synthesis from the second universal primer is performed. If desired, the process may be repeated for a third and any subsequent primer/target region pair. In preferred embodiments, template nucleic acid molecules are single-stranded and all primers are hybridized to the same strand of a template nucleic acid. Template nucleic acid may be immobilized on a solid support, for example, with the 3′-end being tethered to the support and the 5′-end being free.

In some embodiments, real-time sequencing by synthesis is used. Real-time single molecule sequencing-by-synthesis involves the detection of fluorescently labeled nucleotides as they are incorporated into a nascent strand of DNA that is complementary to the template being sequenced. In some embodiments, only one species of the labeled nucleotide is added at a time, and its location in the growing chain is detected. The sequential addition of all four labeled nucleotides is referred to as “quad.” Due to a less-than-100% incorporation efficiency, some nucleotide chains may grow slower than others. Thus, to allow slow-growing chains to “catch-up” so that the first-target sequence is fully read in the first sequencing round, the first target sequence and the second universal primer sites may be separated by a “stalling” nucleotide spacer, i.e., a short nucleotide sequence having a significantly lower incorporation rate per “quad” as compared to the target sequences. Examples of such spacers include homopolymeric nucleotide spacers that are 4-20 nt long.

Accordingly, in particular embodiments, the invention provides a method of sequencing a nucleic acid molecule that includes the steps of:
a) obtaining the plurality of template nucleic acid molecules, wherein each of the nucleic acids comprises i) through v) below arranged in the 3′-to-5′ direction:
   i) the complement of the first universal primer,
   ii) a sample-specific barcode sequence (e.g., a well barcode),
   iii) a homopolymeric nucleotide spacer,
   iv) the complement of the second universal primer, and
   v) a gene-specific barcode sequence (e.g., a gene barcode);
b) hybridizing the first universal primer to the plurality of nucleic acid molecules;
c) performing sequencing by synthesis by elongating the first universal primer thereby identifying the first barcode sequence;
d) incorporating a chain-terminating nucleotide;
e) hybridizing the second universal primer to the plurality of nucleic acid molecules; and
f) performing sequencing by synthesis by elongating the second universal primer thereby identifying the second barcode sequence.

### BRIEF DESCRIPTION OF THE FIGURES

FIG. 1 depicts one illustrative embodiment of the invention. Barcoded nucleic acids are first captured onto a solid support at the 3′ end by hybridization to a capture sequence/first primer (step 1). Next, the first barcode (well barcode (WBC)) is sequenced by synthesis (step 2). The short spacer sequence after the first barcode buffers the second sequencing primer site from base additions during first round sequencing thereby enabling slow barcodes to catch up to all others without inhibiting second round sequencing. After sequencing the first barcode, WBC, terminating nucleotides (ddNTPs) are added to stop the first round sequencing (step 3). Subsequently, the second sequencing primer is hybridized to the template in an optimized reaction (step 4) and sequencing recommences from the second primer into the second barcode (step 5). The hybridization efficiency for the second primer can be monitored using a dye-labeled primer (depicted by a dark circle).

FIG. 2 provides an overview of a barcoding method for GE-HTS. Two oligonucleotide probes are designed against each transcript of interest. The first probe contains a first universal primer site and a target gene-specific sequence (˜10-50 nt). The second probe contains the second target gene-specific sequence (˜10-50 nt), a gene-specific barcode (GBC), and a GBC universal primer site, distinct from the site on the first probe. mRNAs (or cDNAs) are captured on immobilized poly-dT. The pre-designed probes are then annealed to captured mRNA (or cDNA) and ligated to create a barcoded strand. The barcoded strand can then be amplified. Next, a second set of two oligonucleotide probes, one of which contains the first universal primer, while the other contains a second barcode (sample/well-specific barcode (WBC), a WBC universal primer sequence and a sequence complementary to the GBC universal primer in the GBC barcoded strand. The mixture of the second set of oligos and annealed probe from step one is subjected to an amplification process (e.g., PCR) to create a contiguous strand containing the two barcodes. The product of this process is then subjected to methods of sequencing by synthesis to analyze the combinations of both barcodes (GBC/WBC) formed.

FIG. 3 illustrates GBC- and WBC-containing oligonucleotides that were used in the procedures described in the Example.

### DETAILED DESCRIPTION OF THE INVENTION

The invention relates to methods of sequencing nucleic acid molecules, such as DNA and RNA, and especially, to methods of sequencing by synthesis on systems with a limited read length (e.g., less than 60-70 nts). In particular, the methods of the invention can be used for sequencing two or more target regions whose combined lengths plus the length of any intermediate sequence exceeds the available read length on a given sequencing platform.

The present invention provides a method of sequencing a nucleic acid molecule that includes two or more target regions, such as, for example, barcodes that provides a rapid and cost effective way to conduct high-throughput gene expression analysis, for example, in screening a large number of compounds and/or genes with the goal of identifying a therapeutically effective compound or to provide insight into the treatment of disease.

The invention utilizes nucleic acid constructs containing at least the following elements i) through v), arranged in the recited order in the 3′-to-5+ direction:
i) a complement of a first universal primer,
ii) a first target sequence,
iii) a polynucleotide spacer (optional),
iv) a complement of a second universal primer, and
v) a second target sequence.

The invention also provides complements of the recited constructs, and reagent kits, comprising such constructs/complements and primers and other oligonucleotides for performing the method of invention.

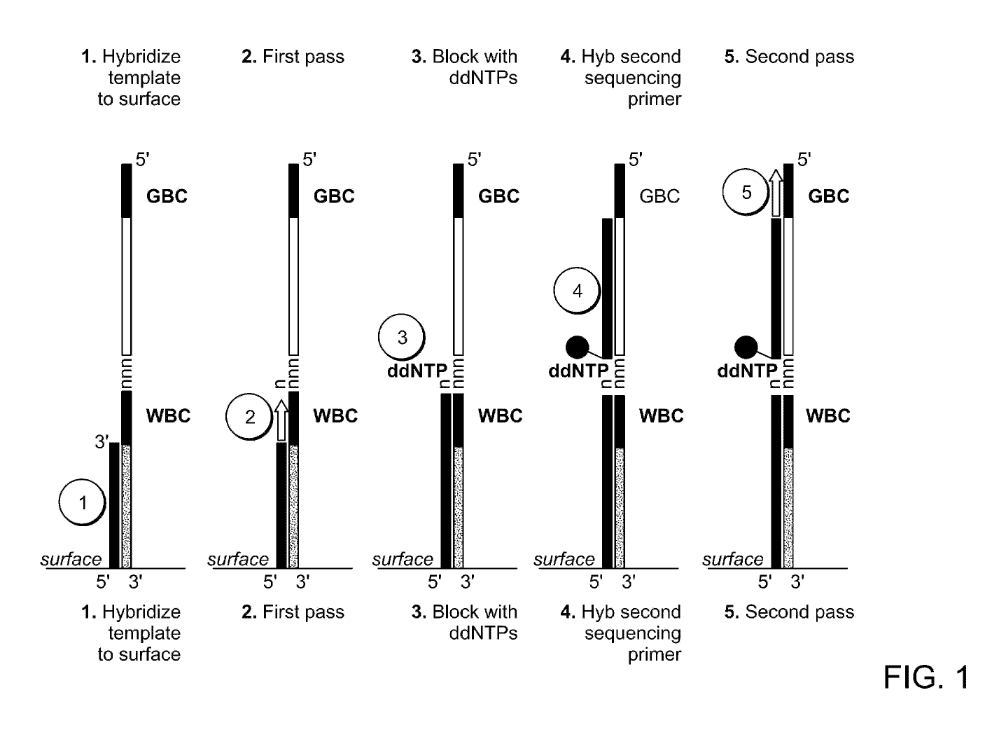

FIG. 1 illustrates an embodiment of the invention that involves the use of barcoded nucleic acids as target sequences. Barcoded nucleic acids are first captured onto a solid support at the 3′ end by hybridization to a capture sequence/first primer (step 1). Further, the first barcode (well barcode (WBC)) is sequenced by synthesis (step 2). The short spacer sequence after the first barcode buffers the second sequencing primer site from base additions during first round sequencing, thereby enabling slow barcodes to catch up to all others without inhibiting second round sequencing. After sequencing the first barcode, WBC, terminating nucleotides (ddNTPs) are added to stop the first round sequencing (step 3). Subsequently, the second sequencing primer is hybridized to the template in an optimized reaction (step 4) and sequencing recommences from the second primer into the second barcode (step 5). The hybridization efficiency for the second primer can be monitored using a dye-labeled primer (depicted by a dark circle).

Accordingly, the invention provides a method of sequencing a nucleic acid molecule that comprises:
a) obtaining a plurality of biological samples, each sample containing a plurality of nucleic acid molecules, wherein each of the nucleic acids comprises i) through v) below, arranged in the recited order in the 3′-to-5′ direction:
   i) a complement of a first universal primer (a first priming site),
   ii) a first target sequence,
   iii) optionally, a polynucleotide spacer,
   iv) a complement of a second universal primer (a second priming site), and
   v) a second target sequence;
b) performing first sequencing by synthesis by extending the first universal primer, thereby sequencing the first target sequence;
c) terminating the sequencing of step b) before the complement of the second primer is reached; and
d) performing second sequencing by synthesis by extending the second universal primer, thereby sequencing the second target sequence.
   In some embodiments, the first and the second universal primers are hybridized sequentially to the plurality of template nucleic acids. For example, as illustrated in FIG. 1, the first universal primer is initially hybridized to the first priming sites in the plurality of nucleic acids. Then, before the growing chain would otherwise extend into the second priming site, the first round of sequencing is terminated, e.g., by addition of a chain-terminating nucleotide (ddNTP, e.g., ddATP, ddTTP, ddCTP, ddUTP, ddGTP, or combination thereof). Any nucleotide triphosphate or analog which lacks a 3′-OH and is a substrate for a polymerase may be used for this process. Following termination, the second universal primer is then hybridized to the second priming sites in the plurality template nucleic acids.

### Target Nucleic Acids, Including Barcodes

In some embodiments, the first target sequence comprises a sample-specific barcode sequence which identifies the source of the sample. The barcode may identify the sample, e.g., by its serial number, source, and/or location during processing (e.g., a plate-specific barcode, a batch-specific barcode, etc.). These barcodes may be indicative of the origin of the sample, different treatment conditions, disease, tissue, etc. For example, the barcode may identify a compound tested in a given sample from a library of compounds. As another example, the barcode may correspond to the source of tissue or cells from a tissue/cell bank.

In some embodiments, the second target sequence comprises a gene-specific barcode sequence which identifies a gene which the nucleic acid is encoded by or from which it is obtained.

Optionally, a third, fourth, fifth, etc., target sequence can be present in the template nucleic acid being analyzed. Each of such target sequences may be separated in manner similar to the first and second target sequences, i.e., with an individual universal priming site, each optionally preceded by a polynucleotide spacer. The third and subsequent barcodes, if any, may identify any of the above parameters, similarly to the first and second barcode. Use of multiple barcodes to encode the identity of a sample may be advantageous as it allows one to reduce the number of starting oligonucleotides. For example, the first barcode may identify the sample position on a plate, while the second barcode may identify the plate number. The exact order of such barcodes relative to each other is not essential.

In general, the term “barcode” refers to known nucleic acid sequences that are specifically added to naturally occurring sequences to serve as unique identifiers of the sequence identity, origin, or source. Examples of barcodes are described, for example, in Shoemaker et al. (1996) Nature Genetics, 14:450; Parameswaran et al. (2007) Nucleic Acids Res., 35:e130; and in the Example. Barcodes are typically less than 20-nucleotides long and are designed to be maximally different yet still retain similar hybridization properties to facilitate simultaneous analysis on high-density oligonucleotide arrays. In some embodiments, a barcode used in the methods of the invention may be, for example, 4-25, 6-18, 8-14, or 10-12 nts long. Desirable barcode sequences have no homopolymers (2 or more of the same base in a row), have sequence edit distances greater than 2 or more bases apart in the encoded barcode (so that the barcodes are error tolerant, i.e., sequencing-by-synthesis process reading errors do not convert a barcode from one to another), and have sequences which are normalized for growth rate in the sequencing-by-synthesis process (ideally, between 1.2-1.6 bases decoded per quad).

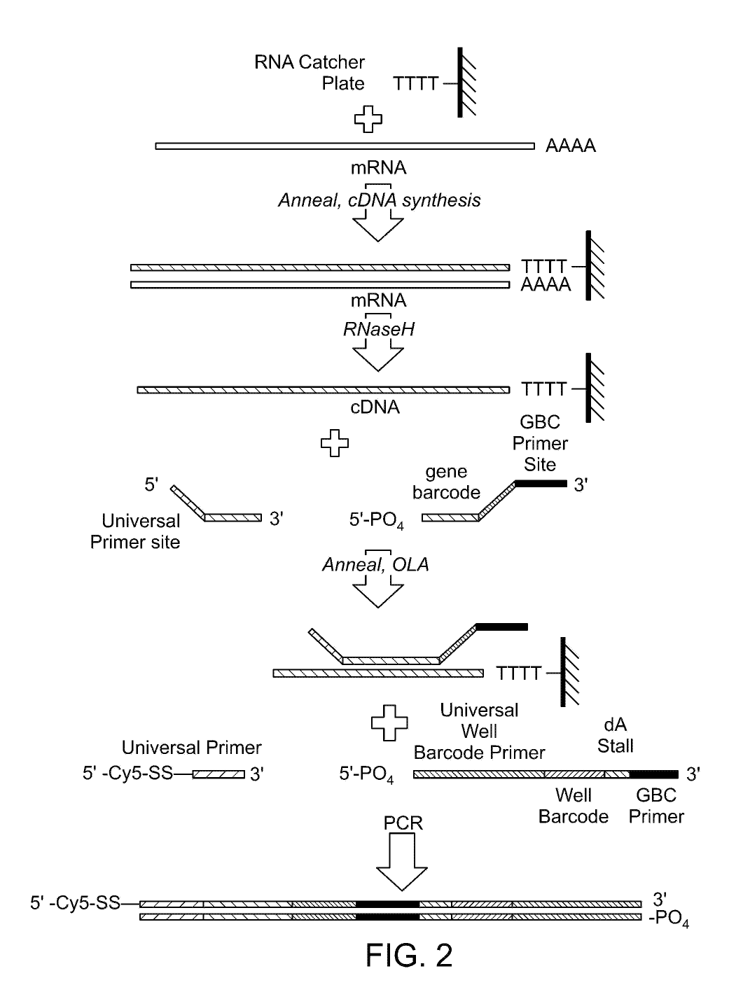

FIG. 2 provides an overview of barcoding for GSS. In brief, two oligonucleotides are designed against each transcript/gene of interest. The first oligonucleotide contains a “Universal Primer site” and a gene-specific half (˜20 nt). The second contains another gene-specific half (˜20 nt), a gene-specific barcode (GBC), and a “GBC primer” site, distinct from the priming site on the first probe. mRNAs (or cDNAs) are captured on immobilized poly-dT (“RNA Catcher Plate”). The pre-designed primers are then annealed to captured mRNA (or cDNA) and ligated to create a barcoded strand. The barcoded strand can be amplified by PCR or another amplification method. Next, a second set of two oligonucleotides, one of which is “Universal Primer”, and the other contains a second barcode (sample/well-specific barcode (WBC)) and a Universal Well Barcode Primer. The second set of probes is then annealed to the barcoded strand and amplified by PCR or another amplification method to create a final strand with the two barcodes. A more detailed explanation of the barcoding procedure is provided in the Example. One of skill in the art may be readily adapted for a wide range of barcodes and other target sequences.

### Universal Primers

DNA polymerases used for sequencing require a primer. A primer is a short, synthetic, single-stranded DNA molecule of known sequence, typically 18-40 bases long, which anneals to its complementary sequence (“priming site”) on the template nucleic acid and allows a polymerase to initiate replication. The term “universal primer,” as used herein, refers to a primer common to a plurality of nucleic acids being analyzed. For example, all or a subset (e.g., 10%, 20%, 30%, 40% 50%, 60%, 70%, 80%, 90%, or more) of all nucleic acids in the sample may share the identical universal priming site, allowing for the simultaneous synthesis of the different nucleic acids in the sample using a single universal primer. In some embodiments, the primers consist of at least 16, 17, 18, 19, 20, 21, 22, 23, 24, 26, 28, 30 or more nucleotides.

Nonlimiting examples of commonly used universal primers can be found in, for example, Messing (2001) Methods Mol. Biol. 167:13-31; and in Alphey, DNA Sequencing (Introduction to BioTechniques), p. 28, Garland Science; 1st edition (1997); see also Table 1 below (note that the exact sequences of the exemplified primers may vary slightly from those shown in the table.). Any number of other suitable primers can be designed by one of skill in the art, using for example, the PROBEWIZ software available at www.cbs.dtu.dk/services/DNAarray/probewiz.php or other tools. In some embodiments, the primers are selected from the primers listed in Table 1 and their complementary sequences. In some embodiments, the primers comprise at least, for example, 16, 17, 18, 19, 20, 21, 22, 23, 24, 26, 28, or 30 nucleotides of any one of the primers listed in Table 1 and their complementary sequences. In some embodiments, the primers are selected from T3 and RG2 (including their complements). In some embodiments, the first and the second primer are less than 70%, 60%, 50%, 40%, 30%, identical to each other.

In some embodiments, the primer may contain a detectable label, e.g., florescent labels such as Cy5 (red) or Cy3 (green), or other labels as described in the General Considerations section. The primer presence of labels aids in determining location of a primer as well as efficiency of primer hybridization. By way of example, the hybridization efficiency for the second primer might be monitored using either a noncleavable green dye on platforms with multicolor capabilities or by a red cleavable dye on the primer for a one-color system.

In general, sets of barcodes and the corresponding primers are developed to minimize self-hybridization into hairpin structures and cross-hybridization with both each other and other components of the reaction mixtures, including the target sequences and sequences on the larger nucleic acid sequences outside of the target sequences (e.g., to sequences within genomic DNA). In addition, the primers designed may be compared to the known sequences in the template nucleic acid, to avoid hybridization of the priming sites and barcodes to gene-derived portions of the nucleic acids. For example, primers and barcodes for use in detecting nucleotides in human genomic DNA can be “BLASTed” against human GenBank sequences, e.g., at www.ncbi.nlm.nih.gov. There are numerous other algorithms that can be used for comparing and analyzing nucleic acid sequences.

Additionally, one of the primers, e.g., the “first primer,” can be used as a universal capture sequence. In such a case, the primer may be covalently bound to a solid support, on which the template nucleic acid is immobilized by hybridization to the primer. (For further details see the description of the universal capture sequences and the Example below.)

TABLE 1
Examples of Universal Primers

| | | |
|---|---|---|
| Primer name | Sequence | SEQ ID NO: |

| | | |
|---|---|---|
| 5′AOX | GACTGGTTCCAATTGACAAG | 1 |
| 3′AOX | GCAAATGGCATTCTGACATCC | 2 |
| BGH reverse | TAGAAGGCACAGTCGAGG | 3 |
| CMV-for | CGCAAATGGGCGGTAGGCGTG | 4 |
| DON1 (forward) | TCGCGTTAACGCTAGCATGGATC | 5 |
| | TC | |
| DON2 (reverse) | GTAACATCAGAGATTTTGAGACAC | 6 |
| EGFP-C | ATGGTCCTGCTGGAGTTC | 7 |
| EGFP-N | CGTCGCCGTCCAGCTCGACCAG | 8 |
| GLprimer1 | TGTATCTTATGGTACTGTAACTG | 9 |
| GLprimer2 | CTTTATGTTTTTGGCGTCTTCC | 10 |
| M13 Forward | GTAAAACGACGGCCAGT | 11 |
| M13 Reverse | CAGGAAACAGCTATGAC | 12 |
| pBAD Forward | ATGCCATAGCATTTTTATCC | 13 |
| pBAD Reverse | GATTTAATCTGTATCAGG | 14 |
| pFastBacF | GGATTATTCATACCGTCCCA | 15 |
| pFastBacR | CAAATGTGGTATGGCTGATT | 16 |
| pGEX 3′ | CCGGGAGCTGCATGTGTCAGAGG | 17 |
| pGEX 5′ | GGGCTGGCAAGCCACGTTTGGTG | 18 |
| pQEPromotor | CCCGAAAAGTGCCACCTG | 19 |
| pQEReverse | GTTCTGAGGTCATTACTGG | 20 |
| pTriplEx 3′ | ACTCACTATAGGGCGAATTG | 21 |
| pTriplEx 5′ | CTCGGGAAGCGCGCCATTGTGTTG | 22 |
| | GT | |
| RV primer3 | CTAGCAAAATAGGCTGTCCC | 23 |
| RV primer4 | GACGATAGTCATGCCCCGCG | 24 |
| S-Tag primer | GAACGCCAGCACATGGACA | 25 |
| SP6 | ATTTAGGTGACACTATA | 26 |
| T3 | ATTAACCCTCACTAAAG | 27 |
| T7 (short) | AATACGACTCACTATAG | 28 |
| T7 (long) | AATACGACTCACTATAGGG | 29 |
| T7 terminator | GCTAGTTATTGCTCAGCGG | 30 |
| RG2 | TCCACTTATCCTTGCATCC | 31 |
| | ATCCTCTGCCCTG | |

### Polynucleotide Spacers

In some embodiments of the invention, real-time sequencing is used. In such embodiments, only one species of the optically labeled nucleotide is added at a time, and its location in the growing chain is detected. Because among the plurality of nucleic acids, various chains may grow at different rates, it might be necessary to allow slow-growing chains to “catch-up” before the first sequencing round is terminated. To that end, the first target sequence and the second universal primer sites can be separated by a “stalling” nucleotide spacer, which is a short nucleotide sequence that has a significantly lower incorporation rate per “quad” as compared to the target sequences. Examples of such spacers includes homopolymeric nucleotide spacers that are, for example, 4-20, 4-16, 4-12, 4-10, 4-8, or 4-6 nts long. However, spacers containing multiple nucleotide species can also be used so long as their “per quad” incorporation rate is lower than that of the first target sequence. In some embodiments, the spacer is selected from polyA, polyC, polyT, polyG, or polyU. In certain embodiments, the spacer is AAAAA. Other mechanisms, such as non-sequencable a basic polynucleotide spacers, can also be also used.

### Sample Preparation

Methods of the invention are particularly suitable for gene expression analysis in high-throughput screens (GE-HTS) that involve assaying multiple samples and multiple gene transcripts. Accordingly, in some embodiments, a plurality of biological samples is obtained, e.g., 24, 96, 384, 1536 or more. The samples may represent different treatment conditions (e.g., test compounds from a chemical library), tissue or cell types, or source (e.g., blood, urine, cerebrospinal fluid, seminal fluid, saliva, sputum, stool), etc. Each of the samples may contain a plurality (e.g., 10, 50, 100, 500, 1000, or more) of nucleic acid constructs in accordance with the present invention. In the case of GE-HTS, each construct may represent a gene transcript whose expression level is being measured.

Nucleic acids to be analyzed may come from a variety of sources. For example, nucleic acids can be naturally occurring DNA or RNA (e.g., mRNA or non-coding RNA) isolated from any source, recombinant molecules, cDNA, or synthetic analogs. For example, nucleic acids may include whole genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), alielic variants, other mutations. Nucleic acids may also include tRNA, rRNA, ribozymes, splice variants, antisense RNA, or siRNA.

Nucleic acids may be obtained from whole organisms, organs, tissues, or cells from different stages of development, differentiation, or disease state, and from different species (human and non-human, including bacteria, fungus, and viral proteins). Various methods for extraction of nucleic acids from biological samples are known (see, e.g., Nucleic Acids Isolation Methods, Bowein (ed.), American Scientific Publishers (2002)). Typically, genomic DNA is obtained from nuclear extracts that are subjected to mechanical shearing to generate random long fragments. For example, genomic DNA may be extracted from tissue or cells using a Qiagen DNeasy Blood & Tissue kit following the manufacturer's protocol. Generally, nucleic acid can be extracted from a biological sample by a variety of techniques such as those described by Maniatis et al., Molecular Cloning: A Laboratory Manual, Cold Spring Harbor, N.Y., pp. 280-281 (1982). Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application Publication 2002/0190663.

### Sequencing, Including Sequencing by Synthesis

Methods of the inventions can be used in the context of sequencing by synthesis. The invention is advantageous for high throughput sequencing platforms, particularly, sequencing by synthesis, where two or more target regions within the same template need to be sequenced. However, their combined lengths plus the length of any intermediate sequence exceeds the available read length on a given sequencing platform.

Four major high-throughput sequencing platforms are currently available: the Genome Sequencers from Roche/454 Life Sciences (Margulies et al. (2005) Nature, 437:376-380; U.S. Pat. Nos. 6,274,320; 6,258,568; 6,210,891), the 1G Analyzer from Illumina/Solexa (Bennett et al. (2005) Pharmacogenomics, 6:373-382), the SOLiD system from Applied Biosystems (solid.appliedbiosystems.com), and the Heliscope system from Helicos Biosciences (see U.S. Patent App. Pub. No. 2007/0070349 and the Example below). Each of these platforms can be used in the methods of the invention. Comparison across the three platforms reveals a trade-off between average sequence read length and the number of DNA molecules that are sequenced. Currently, the average read lengths on these major platforms are as follows: Roche/454, 250 nts (depending on the organism); Illumina/Solexa, 25 nts; SoliD, 35 nts; Heliscope, 25 nts. Thus, in some embodiments, the sequencing platforms used in the methods of the present invention have one or more of the following features:
1) the average available read length is 50, 40, 30, 25, or 20 or fewer nucleotides;
2) four differently optically labeled nucleotides are utilized (e.g., 1G Analyzer);
3) sequencing-by-ligation is utilized (e.g., SOLiD);
4) pyrophosphate detection is utilized (e.g., Roche/454); and
5) four identically optically labeled nucleotides are utilized (e.g., Helicos).

In some embodiments, the invention provides a method of determining a nucleic acid copy number, comprising capturing an unamplified target nucleic acid onto a solid surface using methods of the invention and determining the number of the captured target nucleic acids, for example, by reference to a known control. Heliscope is the only one of the four systems that provides true single-molecule sequencing (tSMS™), thus eliminating amplification artifacts such as errors or bias. Thus, in some embodiments, the methods of the invention are practiced on tSMS™ system.

In some embodiments, a plurality of nucleic acid molecules being sequenced is bound to a solid support. To immobilize the nucleic acid on a solid support, a “capture sequence” can be added, for example, at the 3′ end of the template. The nucleic acids are bound to the solid support by hybridizing the capture sequence to a complementary sequence covalently attached to the solid support. The capture sequence, also referred to as a universal capture sequence, is a nucleic acid sequence complimentary to a sequence attached to a solid support that may also serve as a universal primer. In some embodiments, the capture sequence is poly Nₙ, wherein N is U, A, T, G, or C, n≧5, e.g., 20-70, 40-60, e.g., about 50. For example, the capture sequence could be polyT₄₀₋₅₀ or its complement.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair.

The solid support may be, for example, a glass surface such as described in, e.g., U.S. Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection/sequencing, e.g., as described in the Example and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide may be incorporated and multiple lasers may be utilized for stimulation of incorporated nucleotides.

Other details and variations of the sequencing methods are provided below.

A. Nucleotides

Nucleotides useful in the invention include any nucleotide or nucleotide analog, whether naturally occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably comprise a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

B. Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al. (1991) Gene, 108:1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh et al. (1977) Biochim. Biophys. Acta, 475:32), Thermococcus litoralis (Tli) DNA polymerase (also referred to as Vent® DNA polymerase, Cariello et al. (1991) Polynucleotides Res., 19:4193; New England Biolabs), 9° Nm® DNA polymerase (New England Biolabs), Stoffel fragment, ThermoSequenase® (Amersham Pharmacia Biotech UK), Therminator® (New England Biolabs), Thermotoga maritima (Tma) DNA polymerase (Diaz et al. (1998) Braz. J. Med. Res., 31:1239), Thermus aquaticus (Taq) DNA polymerase (Chien et al. (1976) J. Bacteoriol., 127: 1550), DNA polymerase, Pyrococcus kodakaraensis KOD DNA polymerase (Takagi et al. (1997) Appl. Environ. Microbiol., 63:4504), JDF-3 DNA polymerase (from thermococcus sp. JCDF-3, PCT Patent Application Publication WO 01/32887), Pyrococcus GB-D (PGB-D) DNA polymerase (also referred as Deep Vent® DNA polymerase, Juncosa-Ginesta et al. (1994) Biotechniques, 16:820; New England Biolabs), UITma DNA polymerase (from thermophile Thermotoga maritima; Diaz et al. (1998) Braz. J. Med. Res., 31:1239; PE Applied Biosystems), Tgo DNA polymerase (from thermococcus gorgonarius, Roche Molecular Biochemicals), E. coli DNA polymerase I (Lecomte et al. (1983) Polynucleotides Res., 11:7505), T7 DNA polymerase (Nordstrom et al. (1981) J. Biol. Chem., 256:3112), and archaeal DP11/DP2 DNA polymerase II (Cann et al. (1998) Proc. Natl. Acad. Sci. USA, 95:14250-5).

While mesophilic polymerases are contemplated by the invention, preferred polymerases are thermophilic. Thermophilic DNA polymerases include, but are not limited to, ThermoSequenase®, 9° N®, Therminator®), Taq, Tne, Tma, Pfu, Tfl, Tth, Tli, Stoffel fragment, Vent® and Deep Vent®0 DNA polymerase, KOD DNA polymerase, Tgo, JDF-3, and mutants, variants and derivatives thereof.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin (1997) Cell, 88:5-8; Verma (1977) Biochim. Biophys. Acta, 473:1-38; Wu et al. (1975) CRC Crit. Rev. Biochem., 3:289-347).

C. Surfaces

In a preferred embodiment, nucleic acid template molecules are attached to a solid support (“substrate”). Substrates for use in the invention can be two-or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Erie Glass (Portsmouth, N.H.). In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the primer to the surface of the substrate. The immobilization can be achieved through direct or indirect bonding to the surface. The bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. A preferred attachment is direct amine bonding of a terminal nucleotide of the template or the primer to an epoxide integrated on the surface. The bonding also can be through non-covalent linkage. For example, biotin-streptavidin (Taylor et al. (1991) J. Phys. D: Appl. Phys., 24:1443,) and digoxigenin with anti-digoxigenin (Smith et al. (1992) Science, 253:1122, are common tools for anchoring nucleic acids to surfaces and parallels. Alternatively, the attachment can be achieved by anchoring a hydrophobic chain into a lipid monolayer or bilayer. Other methods known in the art for attaching nucleic acid molecules to substrates can also be used.

### D. Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include the scanning tunneling microscope (siM) and the atomic force microscope (AFM). Hybridization patterns may also be scanned using a CCD camera (e.g., Model TEICCD512SF, Princeton Instruments, Trenton, N.J.) with suitable optics (Ploem, in Fluorescent and Luminescent Probes for Biological Activity, Mason (ed.), Academic Press, Landon, pp. 1-11 (1993), such as described in Yershov et al. (1996) Proc. Natl. Acad. Sci., 93:4913, or may be imaged by TV monitoring. For radioactive signals, a Phosphorlmager™ device can be used (Johnston et al. (1990) Electrophoresis, 13:566; Drmanacetal. (1992) Electrophoresis, 13:566). Other commercial suppliers of imaging instruments include General Scanning Inc., (Watertown, Mass.; genscan.com), Genix Technologies (Waterloo, Ontario, Canada; confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

The following Example provides illustrative embodiments of the invention and does not in any way limit the invention.

### EXAMPLE

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated oligonucleotide (TCCACTTATCCTTGCATCCATCCTCTGCCCTG (SEQ ID NO:32)) is incubated with each slide for 30 minutes at room temperature in a volume of 80 ml. The slides are then treated with phosphate (1 M) for 4 hours at room temperature in order to passivate the surface. Slides are then stored in 20 mM Tris, 100 mM NaCl, 0.001% Triton X-100, pH 8.0 at 4° C. until they are used for sequencing.

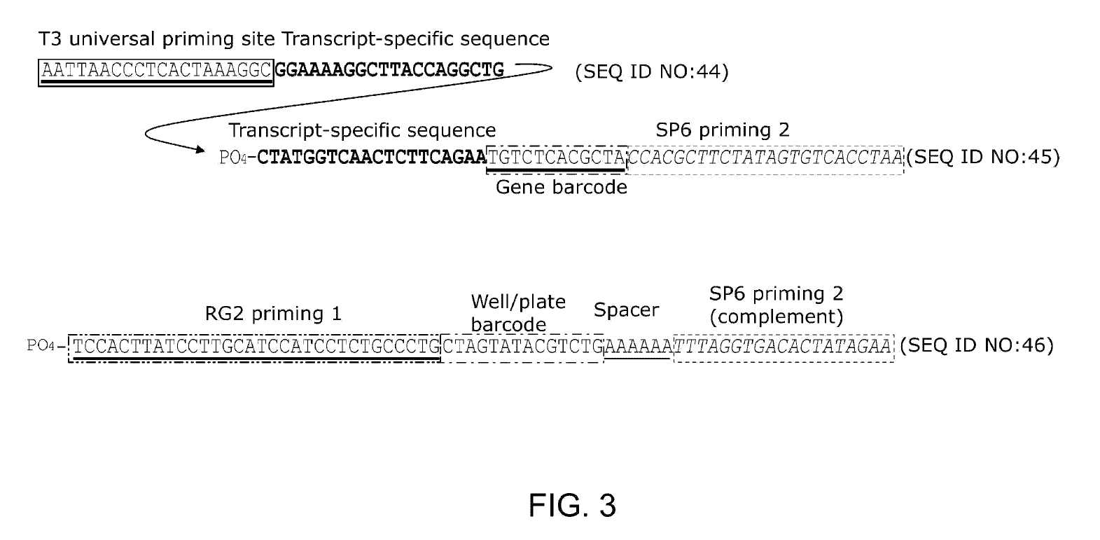

For sequencing, the slide is placed in a modified FCS2 flow cell (Bioptechs, Butler, Pa.) using a 50-μm thick gasket. The flow cell is placed on a movable stage that is part of a high-efficiency fluorescence imaging system built based on a Nikon TE-2000 inverted microscope equipped with a total internal reflection (TIR) objective. The slide is then rinsed with HEPES buffer with 100 mM NaCl and equilibrated to a temperature of 50° C. An aliquot of the synthetic oligonucleotides (examples of sequences are provided as SEQ ID NOs:33-42 and in FIG. 3) designed to mimic the PCR product of the Genome Signature Sequencing (GSS™) process is diluted in 3×SSC to a final concentration of 200 pM (each). A 100-μl aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains tens of thousands of GSS™ oligonucleotide/primer template duplexes randomly bound to the glass surface. The temperature of the flow cell is then reduced to 37° C. for sequencing and the objective is brought into contact with the flow cell.

Further, cytosine triphosphate, guanidine triphosphate, adenine triphosphate, and uracil triphosphate, each having a cleavable cyanine-5 label (at the 7-deaza position for ATP and GTP and at the C5 position for CTP and UTP (PerkinElmer)) are stored separately in buffer containing 20 mM Tris-HCl, pH 8.8, 50 μM MnSO₄, 10 mM (NH4)₂SO₄, 10 mM HCl, and 0.1% Triton X-100, and 50 U Klenow exo⁻ polymerase (NEB). Sequencing proceeds as follows.

First, initial imaging is used to determine the positions of DNA duplexes on the epoxide surface. The Cy3 label attached to the synthetic oligo fragments is imaged by excitation using a laser tuned to 532 nm radiation (Verdi V-2 Laser, Coherent, Santa Clara, Calif.) in order to establish duplex position. For each slide only single fluorescent molecules that are imaged in this step are counted. Imaging of incorporated nucleotides as described below is accomplished by excitation of a cyanine-5 dye using a 635-nm radiation laser (Coherent). 100 nM Cy5-CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 μl volumes each, followed by 150 mM HEPES/150 mM NaCl/pH 7.0 (“HEPES/NaCl”) (10 times at 60 μl volumes). An oxygen scavenger containing 30% acetonitrile and scavenger buffer (134 μl 150 mM HEPES/100 mMNaCl, 24 μl 100 mM Trolox in 150 mM MES, pH 6.1, 10 μl 100 mM DABCO in 150 mM MES, pH 6.1, 8 μl 2M glucose, 20 μl 150 mM Nal, and 4 μl glucose oxidase (USB) is next added. The slide is then imaged (100 frames) for 250 milliseconds using an Inova 301K laser (Coherent) at 647 nm, followed by green imaging with a Verdi V-2 laser (Coherent) at 532 nm for 500 milliseconds to confirm duplex position. The positions having detectable fluorescence are recorded. After imaging, the flow cell is rinsed 5 times each with SSC/HEPES/SDS (60 μ) and HEPES/NaCl (60 μl). Next, the cyanine-5 label is cleaved off incorporated CTP by introduction into the flow cell of 50 mM TCEP/250 mM Tris, pH 7.6/100 mM NaCl for 5 minutes, after which the flow cell is rinsed 5 times each with SSC/HEPES/SDS (60 μl) and HEPES/NaCl (60 μl). The remaining nucleotide is capped with 50 mM iodoacetamide/100 mM Tris, pH 9.0/100 mM NaCl for 5 minutes followed by rinsing 5 times each with SSC/HEPES/SDS (60 μl) and HEPES/NaCl (60 μl). The scavenger is applied again in the manner described above, and the slide is again imaged to determine the effectiveness of the cleave/cap steps and to identify non-incorporated fluorescent objects.

The procedure described above is then conducted with 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of 40 cycles.

Once the desired number of cycles is completed, the image stack data (i.e., the single-molecule sequences obtained from the various surface-bound duplex) are aligned to the reference barcode sequences. The individual single molecule sequence read lengths obtained range from 2 to 16 consecutive nucleotides with about 12.6 consecutive nucleotides being the average length and only those greater than 9 bases in length with less than 2 errors where used in the final analysis.

The sequencing products of the first barcode are terminated using 10 μM ddNTPs and Therminator™ (NEB) for 15 min at 45° using Therminator™ buffer provided by the manufacturer. The flow cell is rinsed using HEPES/0.5 M NaCl to remove the polymerase and ddNTPs from the system. Additional rinses are performed with standard HEPES/NaCl.

The second primer (CGACATCGCACGAATAGACGGCACTCAGAC (SEQ ID NO:43)) which has a 5′-cleavable Cy5 is diluted in 3×SSC to a final concentration of 1 nM. A 100-μl aliquot is placed in the flow cell and incubated on the slide for 15 minutes at 37° C. After incubation, the flow cell is rinsed with 1×SSC/HEPES/0.1% SDS followed by HEPES/NaCl. A passive vacuum apparatus is used to pull fluid across the flow cell.

The sequencing process is repeated as previously described except the first picture taken is a red image since the second primer is labeled with a cleavable Cy5 dye. Following imaging, the cleavable red dye is removed and capped using TCEP and iodoacetamide solutions and cycles of C, U, A, and G are performed as previous (40 total cycles).

Once the desired number of cycles is completed, the image stack data (i.e., the single-molecule sequences obtained from the various surface-bound duplex) are aligned to the reference sequence. The individual single molecule sequence read lengths obtained range from 2 to 16 consecutive nucleotides with about 12.6 consecutive nucleotides being the average length and only those greater than 9 bases in length with less than 2 errors are used in the final analysis.

Other details of the protocol are described in process as described, for example, in U.S. Patent Application Publication Nos. 2007/0070349 and 2006/0252077.

TABLE 2

| Step | Efficiency | Overall Yield |
|---|---|---|
| 1^{st} pass 2+ nt reads | 48% of all green | “100%” |
| Sequence out to end | 60% | 60% |
| of 1^{st} barcode | | |
| ddNTP blocking | 98.2% | 59% |
| 2^{nd} template hyb. | 82% | 48% |
| Growth to end | 82% | 40% |
| of 2^{nd} barcode | | |

Representative experimental results for stepwise efficiencies of each step performed essentially as described are shown above. Of all the initial green (template) spots observed, 48% were shown to add the first 2 bases. These strands are defined as the starting pool and set at 100% Overall Yield. After 40 cycles of sequencing, 60% of the individual sequence molecule reads were found to be equal to or greater than the length of barcode one. The efficiency of ddNTP blocking was found to be ˜98%. The efficiency of hybridization of the second primer onto spots with activity during sequencing from the first primer was 82%. After 40 cycles of sequencing, 82% of the reads were found to be equal to or greater than the length of barcode two. The Overall Yield of the entire process is approximately 40% of the initially available templates.

All publications, patents, patent applications, and biological sequences cited in this disclosure are incorporated by reference in their entirety.

1. A method of sequencing a nucleic acid molecule, the method comprising:
a) obtaining a plurality of biological samples, each sample containing a plurality of template nucleic acid molecules, each of the template nucleic acids comprising i) through v) arranged in the recited order in the 3′-to-5′ direction:
i) a complement of a first universal primer,
ii) a first target sequence,
iii) optionally, a polynucleotide spacer,
iv) a complement of a second universal primer, and
v) a second target sequence;
b) performing first sequencing by synthesis by extending the first universal primer, thereby sequencing the first target sequence;
c) terminating the sequencing of step b) before the complement of the second primer is reached; and
d) performing second sequencing by synthesis by extending the second universal primer thereby sequencing the second target sequence.

2. The method of claim 1, wherein the template nucleic acids are single-stranded.

3. The method of claim 1, wherein each of the nucleic acids comprises iii) a polynucleotide spacer.

4. The method of claim 3, wherein the nucleotide spacer is a homopolymer.

5. The method of claim 1, comprising:
hybridizing the first universal primer to the plurality of template nucleic acid molecules prior to step b); and
hybridizing the second universal primer to at least some of the plurality of template nucleic acid molecules following step c).

6. The method of claim 1, wherein the first target sequence comprises a sample-specific barcode sequence which identifies the source of the sample.

7. The method of claim 1, wherein the second target sequence comprises a gene-specific barcode sequence which identifies a gene which the nucleic acid is encoded by or from which it is obtained.

8. The method of claim 1, wherein the sequencing of step b) is terminated by incorporating a chain-terminating nucleotide.

9. The method of claim 1, comprising:
a) obtaining the plurality of template nucleic acid molecules, each of the template nucleic acids comprising i) through v) arranged in the recited order in the 3′-to-5′ direction:
i) the complement of the first universal primer,
ii) a sample-specific barcode sequence,
iii) a homopolymeric nucleotide spacer,
iv) the complement of the second universal primer, and
v) a gene-specific barcode sequence;
b) hybridizing the first universal primer to the plurality of nucleic acid molecules;
c) performing sequencing by synthesis off the first universal primer thereby identifying the first bar code sequence;
d) incorporating a chain-terminating nucleotide;
e) hybridizing the second universal primer to the plurality of nucleic acid molecules; and
f) performing sequencing by synthesis off the second universal primer thereby identifying the second barcode sequence.

10. The method of claim 1, wherein the plurality of template nucleic acid molecules is immobilized a solid support.

11. The method of claim 10, wherein the template nucleic acid molecules are immobilized through their 3′ ends.

12. The method of claim 3, wherein the spacer contains at least 4 but no more than 20 sequential nucleotides of the same nucleotide species.

13. The method of claim 9, further comprising determining a copy number of the template nucleic acid molecules having the same first barcode sequences and the same second barcode sequences.

14. The method of claim 1, wherein the available average read length of the sequence-by-synthesis is less than 50 nucleotides.

15. The method of claim 1, wherein each sample comprises at least 1,000 nucleic acids.

16. The method of claim 9, wherein the sample-specific barcode sequence and the second gene-specific barcode contain no more than 30 nucleotides each.

17. The method of claim 1, wherein the plurality of template nucleic acids are individually optically resolvable while sequenced.

18. The method of claim 1, wherein the first primer serves as a universal capture sequence.

19. The method of claim 1, wherein the capture sequence comprises Nₙ, wherein N is U, A, T, G, or C, and n≧5.

20. The method of claim 13, wherein the second primer contains a detectable label.

21. The method of claim 1, wherein the sequences of the first and the second primers are less than 70% identical.

22. The method of claim 1, wherein the template nucleic acid further comprises a third target sequence which is a plate-specific barcode.

23. A composition comprising a plurality of single-stranded template nucleic acid molecules, wherein each of the nucleic acids comprises:
a) i) through v) arranged in the recited order in the 3′-to-5′ direction:
i) a complement of a first universal primer,
ii) a first target sequence,
iii) a homopolymeric nucleotide spacer,
iv) a complement of a second universal primer, and
v) a second target sequence; and/or
b) a complement of a).

24. The composition of claim 23, wherein the plurality of the template nucleic acid molecules is bound to a solid support at the 3′ end of a) or the 5′ end of b).

25. The composition of claim 23, wherein the first target sequence comprises a sample-specific barcode sequence which identifies the source of the sample, and the second target sequence comprises a gene-specific barcode sequence which identifies a gene which the nucleic acid is encoded by or from which it is obtained.


SEQUENCE LISTING (46 sequences)

SEQ ID NO: 1 - DNA, 20 bp, unknown
  Synthetic oligonucleotide
gactggttccaattgacaag

SEQ ID NO: 2 - DNA, 21 bp, unknown
  Synthetic oligonucleotide
gcaaatggcattctgacatcc

SEQ ID NO: 3 - DNA, 18 bp, unknown
  Synthetic oligonucleotide
tagaaggcacagtcgagg

SEQ ID NO: 4 - DNA, 21 bp, unknown
  Synthetic oligonucleotide
cgcaaatgggcggtaggcgtg

SEQ ID NO: 5 - DNA, 25 bp, unknown
  Synthetic oligonucleotide
tcgcgttaacgctagcatggatctc

SEQ ID NO: 6 - DNA, 24 bp, unknown
  Synthetic oligonucleotide
gtaacatcagagattttgagacac

SEQ ID NO: 7 - DNA, 18 bp, unknown
  Synthetic oligonucleotide
atggtcctgctggagttc

SEQ ID NO: 8 - DNA, 22 bp, unknown
  Synthetic oligonucleotide
cgtcgccgtccagctcgaccag

SEQ ID NO: 9 - DNA, 23 bp, unknown
  Synthetic oligonucleotide
tgtatcttatggtactgtaactg

SEQ ID NO: 10 - DNA, 22 bp, unknown
  Synthetic oligonucleotide
ctttatgtttttggcgtcttcc

SEQ ID NO: 11 - DNA, 17 bp, unknown
  Synthetic oligonucleotide
gtaaaacgacggccagt

SEQ ID NO: 12 - DNA, 17 bp, unknown
  Synthetic oligonucleotide
caggaaacagctatgac

SEQ ID NO: 13 - DNA, 20 bp, unknown
  Synthetic oligonucleotide
atgccatagcatttttatcc

SEQ ID NO: 14 - DNA, 18 bp, unknown
  Synthetic oligonucleotide
gatttaatctgtatcagg

SEQ ID NO: 15 - DNA, 20 bp, unknown
  Synthetic oligonucleotide
ggattattcataccgtccca

SEQ ID NO: 16 - DNA, 20 bp, unknown
  Synthetic oligonucleotide
caaatgtggtatggctgatt

SEQ ID NO: 17 - DNA, 23 bp, unknown
  Synthetic oligonucleotide
ccgggagctgcatgtgtcagagg

SEQ ID NO: 18 - DNA, 23 bp, unknown
  Synthetic oligonucleotide
gggctggcaagccacgtttggtg

SEQ ID NO: 19 - DNA, 18 bp, unknown
  Synthetic oligonucleotide
cccgaaaagtgccacctg

SEQ ID NO: 20 - DNA, 19 bp, unknown
  Synthetic oligonucleotide
gttctgaggtcattactgg

SEQ ID NO: 21 - DNA, 20 bp, unknown
  Synthetic oligonucleotide
actcactatagggcgaattg

SEQ ID NO: 22 - DNA, 26 bp, unknown
  Synthetic oligonucleotide
ctcgggaagcgcgccattgtgttggt

SEQ ID NO: 23 - DNA, 20 bp, unknown
  Synthetic oligonucleotide
ctagcaaaataggctgtccc

SEQ ID NO: 24 - DNA, 20 bp, unknown
  Synthetic oligonucleotide
gacgatagtcatgccccgcg

SEQ ID NO: 25 - DNA, 19 bp, unknown
  Synthetic oligonucleotide
gaacgccagcacatggaca

SEQ ID NO: 26 - DNA, 17 bp, unknown
  Synthetic oligonucleotide
atttaggtgacactata

SEQ ID NO: 27 - DNA, 17 bp, unknown
  Synthetic oligonucleotide
attaaccctcactaaag

SEQ ID NO: 28 - DNA, 17 bp, unknown
  Synthetic oligonucleotide
aatacgactcactatag

SEQ ID NO: 29 - DNA, 19 bp, unknown
  Synthetic oligonucleotide
aatacgactcactataggg

SEQ ID NO: 30 - DNA, 19 bp, unknown
  Synthetic oligonucleotide
gctagttattgctcagcgg

SEQ ID NO: 31 - DNA, 32 bp, unknown
  Synthetic oligonucleotide
tccacttatccttgcatccatcctctgccctg

SEQ ID NO: 32 - DNA, 32 bp, unknown; identical to SEQ ID NO: 31
  Synthetic oligonucleotide

SEQ ID NO: 33 - DNA, 124 bp, unknown
  Synthetic oligonucleotide
gctcactccgggtgtctgggcttttggtttgtggggagcatgtagtgtctgagtgccgtctattcgtgcgatgtcgaaaaaaagcatagatgcagggcagaggatggatgcaaggataagtgga

SEQ ID NO: 34 - DNA, 124 bp, unknown
  Synthetic oligonucleotide
gctcactccgggtgtctgggcttttggtttgtgggacagcacgtctgtctgagtgccgtctattcgtgcgatgtcgaaaaaacgagtgctagcagggcagaggatggatgcaaggataagtgga

SEQ ID NO: 35 - DNA, 124 bp, unknown
  Synthetic oligonucleotide
gctcactccgggtgtctgggcttttggtttgtgggactctcatgctgtctgagtgccgtctattcgtgcgatgtcgaaaaaacagtgtagcgcagggcagaggatggatgcaaggataagtgga

SEQ ID NO: 36 - DNA, 124 bp, unknown
  Synthetic oligonucleotide
gctcactccgggtgtctgggcttttggtttgtgggcatacgtacacgtctgagtgccgtctattcgtgcgatgtcgaaaaaacgagatcagtcagggcagaggatggatgcaaggataagtgga

SEQ ID NO: 37 - DNA, 124 bp, unknown
  Synthetic oligonucleotide
gctcactccgggtgtctgggcttttggtttgtgggtactcagtctagtctgagtgccgtctattcgtgcgatgtcgaaaaaaatatgctgatcagggcagaggatggatgcaaggataagtgga

SEQ ID NO: 38 - DNA, 124 bp, unknown
  Synthetic oligonucleotide
gctcactccgggtgtctgggcttttggtttgtgggtactactgatagtctgagtgccgtctattcgtgcgatgtcgaaaaaacagatcgtctcagggcagaggatggatgcaaggataagtgga

SEQ ID NO: 39 - DNA, 124 bp, unknown
  Synthetic oligonucleotide
gctcactccgggtgtctgggcttttggtttgtggggcgagtcagtagtctgagtgccgtctattcgtgcgatgtcgaaaaaatgagagtacacagggcagaggatggatgcaaggataagtgga

SEQ ID NO: 40 - DNA, 124 bp, unknown
  Synthetic oligonucleotide
gctcactccgggtgtctgggcttttggtttgtgggtctagtagcgtgtctgagtgccgtctattcgtgcgatgtcgaaaaaatcgtacatgccagggcagaggatggatgcaaggataagtgga

SEQ ID NO: 41 - DNA, 124 bp, unknown
  Synthetic oligonucleotide
gctcactccgggtgtctgggcttttggtttgtgggtcagagctagtgtctgagtgccgtctattcgtgcgatgtcgaaaaaactgctacgtccagggcagaggatggatgcaaggataagtgga

SEQ ID NO: 42 - DNA, 124 bp, unknown
  Synthetic oligonucleotide
gctcactccgggtgtctgggcttttggtttgtggggatcacgatgtgtctgagtgccgtctattcgtgcgatgtcgaaaaaacagtctgtaccagggcagaggatggatgcaaggataagtgga

SEQ ID NO: 43 - DNA, 30 bp, unknown
  Synthetic oligonucleotide
cgacatcgcacgaatagacggcactcagac

SEQ ID NO: 44 - DNA, 40 bp, unknown
  Synthetic oligonucleotide
aattaaccctcactaaaggcggaaaaggcttaccaggctg

SEQ ID NO: 45 - DNA, 40 bp, unknown; identical to SEQ ID NO: 44
  Synthetic oligonucleotide

SEQ ID NO: 46 - DNA, 71 bp, unknown
  Synthetic oligonucleotide
tccacttatccttgcatccatcctctgccctgctagtatacgtctgaaaaaatttaggtgacactatagaa